(19) United States
(12) Patent Application Publication
(10) Pub. No.: US 2008/0107662 A1
(43) Pub. Date: May 8, 2008

(54) Immunogenic Peptides, Nucleic Acids Encoding The Same And Use Thereof In Cancer Treatment And Diagnosis

(76) Inventors: Vito Michele Fazio, Roma (IT); Gisella Volpe, Frossasco (IT); Giuseppe Saglio, Torino (IT)

Correspondence Address: YOUNG & THOMPSON, 209 Madison Street, Suite 500, ALEXANDRIA, VA, 22314, US

(21) Appl. No.: 11/632,275

(22) PCT Filed: Jul. 15, 2005
(86) PCT No.: PCT/EP05/07709

§ 371 Date: Apr. 6, 2007

(30) Foreign Application Priority Data
Jul. 16, 2004 (IT) MI2004A001435

(51) Int. Cl. A61K 31/70; C07K 16/00; C07H 21/04; C12N 15/00; C12N 5/00; A01N 63/00; A61P 37/00; A61K 38/00; A61K 39/00; C12N 5/04; C12N 5/02

(52) U.S. Cl. 424/184.1; 530/300; 530/350; 536/23.5; 435/320.1; 435/325; 530/387.7; 514/12; 435/375; 435/372.3; 514/44; 424/93.7

(57) ABSTRACT

The invention discloses alternative splice variants in Philadelphia chromosome positive leukemia, immunogenic peptides and proteins thereby produced and the use thereof in the preparation of antitumor agents.

The present invention relates to immunogenic peptides and proteins useful for the diagnosis, prevention and therapy of tumors, in particular leukemias. The tumor-specific antigens are generated by BCR/ABL alternative splicing in Philadelphia chromosome positive leukemia and display unique immunogenic sequences. The invention further provides pharmaceutical compositions containing the tumor-specific antigens and methods for stimulating an immune response against tumors, or for monitoring tumor progression, using the immunogenic peptides or proteins.

### BACKGROUND OF THE INVENTION

Philadelphia chromosome (Ph) represents the most frequent cytogenetic defect found in human leukemias. Despite the low molecular variability of Ph genetic alterations, many different clinical and hematologic conditions are associated therewith, including mieloproliferative chronic diseases such as chronic myelogenous leukemia (CML) and acute lymphoblastic leukemia (ALL), which are very aggressive and characterized by extremely negative prognosis.

The Ph chromosome positive leukemias are characterized by the (t9;22)(q34;q11) translocation, which gives rise to a diversity of Bcr/Abl transcripts and hybrid fusion proteins. Specifically, a portion of the Abl protooncogene located on chromosome 9 is fused to a portion of Bcr gene on chromosome 22.

The breakpoint on chromosome 9 is located within a large 200 Kb region at the 5′ end of Abl gene, leaving exons 2-11 in the fusion protein, while the breakpoint within the BCR gene extends through chromosome 22 (FIG. 1).

In the majority of CML patients and in about ⅓ of LLA Ph+ patients, the breakpoint is placed in a 5.8 Kb region within the BCR gene, including exons 12-16 (originally indicated as b1-b5), known as the main breaking cluster region (M-Bcr). As a result of alternative splicing, the recombination with c-Abl gene may give rise to two fusion transcripts (b3a2 or b2a2) depending on whether the Abl exon 2 is fused to BCR exon 14 (b3) or 13 (b2). Such transcripts encode a chimeric protein of approx. 210 Kd (p210), which plays a major role in CML pathogenesis due to an extremely potentiated tyrosine-kinase activity.

In some ALL patients and rarely in CML patients the breakpoint is placed in a 5′-region of the BCR gene, between the alternative exons e2′ and e2, known as minor breaking cluster region (m-Bcr). The resulting transcript e1a2 encodes a protein of about 190 Kd (p190). A third breakpoint (μ-Bcr) was found downstream of BCR exon 19 and is involved in the production of a fusion protein of about 230 Kd (p230) associated with the rare Ph+ chronic neutrophilic leukemia.

Different splice variants such as b2a3, b3a3, e1a3, e6a2, e8a2, e2a2 have also been reported. In most cases the recombination involves the second and, in few cases, the third exon of the 3′-ABL gene.

The amino and carboxy terminal regions in the BCR/ABL chimeric fusion proteins and in the respective parent proteins are identical, while the junctions between the two proteins display unique amino acid sequences which are not found in normal cells and therefore may have immunogenic potential. For this reason the immunotherapy approaches to LMC so far experienced have been based on antigenic determinants derived from the unique junctional sequences of BCR/ABL hybrid proteins which are not found in normal cells.

An essential requirement for triggering an immune response against Ph-positive leukemic clones is that the antigens are presented in association with HLA molecules, allowing for immunologic recognition by MHC-restricted T lymphocytes.

Some peptides embracing the Bcr-Abl junction proved able to stimulate in vitro CD4 and CD8-mediated responses. Three phase I and II clinical trials (22,23,7) using b3a2 peptide vaccine have started. A recent study (Vaccination of patients with chronic myelogenous leukemia with BCR-ABL oncogene breakpoint fusion peptides generates specific immune responses Blood 2000, vol. 95 (5), pages 1781-1787), using patients with persistent stable disease during conventional treatment, shows a clinical response to peptide vaccination with seven of 15 complete cytogenetic response. However, not all patients are eligible for peptide vaccination: in fact, because of a poor immunogenicity of sequence p210-b2a2 and p190-e1a2, only specific CML patients with p210-b3a2 and some specific HLA class I or II molecules have been recruited in that trial.

### DESCRIPTION OF THE INVENTION

The invention regards novel tumor antigens resulting from alternative splicing of BCR/ABL-fusion genes. According to a first embodiment, the invention provides a tumor-associated protein comprising an “out of frame” (OOF) amino acid sequence (SEQ ID NO: 1), which is encoded by a BCR/ABL splice variant involving exons 4 and 5 of the ABL gene and exons 1, 13 and 14 of the BCR gene. In a preferred embodiment, the amino-terminal region of the tumor-associated protein consists of an amino acid sequence encoded by one or more exons of the BCR gene, preferably exons 1, from 1 to 13 and from 1 to 14, whereas the carboxy-terminal region consists of SEQ ID NO: 1. The tumor-associated proteins having an amino acid sequence selected from SEQ ID NOs: 2, 3 and 4 are more preferred.

In a further embodiment, the invention provides a tumor-specific peptide consisting of SEQ ID NO: 1 or an immunogenic fragment thereof. The fragments of SEQ ID NO: 1, which are preferably from 9 to 14 amino acids in length, provide T-cell specific epitopes which can be selected by affinity for HLA alleles. The peptides of SEQ ID NOs: 5, 6 and 7, showing affinity for HLA-0201, HLA-03, HLA-B2705, HLA-B0702 and HLA-B5101, represent preferred T-cell epitopes. More preferred are the peptides selected from SEQ ID NO: 12, 13 and 14, showing affinity for HLA-A0201, and the peptides selected from SEQ ID NO: 15, 16 and 17, which are specific for the HLA-A3 allele. Besides displaying binding affinity for HLA molecules, the peptides of the invention, particularly those of SEQ ID NOs: 12-17 and SEQ ID NO: 9, proved able to induce the secretion of cytokines by antigen-specific T-cells.

The immunogenic peptides of the invention can be prepared according to different procedures. For example, they can be chemically synthesized following known procedures (see for example Stewart and Young, (1984) Solid Phase Peptide Synthesis, 2nd ed. Pierce Chemical Co.; Tam et al., J. Am. Chem. Soc. (1983) 105:6442; Merrifield (1979), The peptides, Gross and Meienhofer, eds NY Academic Press, 1-284). The synthesis can be carried out in solution or in solid phase or using an automated synthesizer. Alternatively, peptides can be prepared using recombinant DNA techniques, as described for example in Sambrook et al., Molecular Cloning, A Laboratory Manual, Cold Spring Harbor Press, Cold Spring Harbor, N.Y. (1982), or in Ausuble et al., Current Protocols in Molecular Biology, John Wiley and Sons, Inc., New York (1987).

One or more amino acid residues in the above identified sequences can be replaced with different residues in D or L configuration, or can be chemically modified, for example by amidation of the carboxyl terminus, binding of lipophilic groups (e.g. fatty acids residues), or by glycosylation or conjugation with other peptides, so as to improve their activity profile, in particular their immunogenicity, selectivity and bioavailability. Furthermore, the peptides can be chemically derivatized on the side chains, for example through modification of the carboxylic groups to give salts, esters or amides, or they can be conjugated to different antigens so as to increase the immune response.

A further aspect of the invention relates to a nucleic acid molecule coding for a protein or peptide according to the invention. A DNA sequence encoding for peptide SEQ ID N. 1 is reported in SEQ ID N. 18 and corresponds to the nucleotide sequence 916-1254 of the gene c-Abl (Gene Bank accession number M14752).

According to a further aspect, the invention relates to expression or cloning vectors bearing said nucleic acid molecule and to eukaryotic or prokaryotic host cells containing them. The DNA molecules, the constructs and vectors thereof, can be used in DNA vaccination protocols (Donnelly J. J. Et al., 1994, The Immunologist 2:1). The DNA preparations used for this purpose can be administered intramuscularly, parenterally or mucosally (PNAS 1986, 83, 9551; WO 90/11092), or they can be adsorbed on gold particles and administered transdermally by means of a biolistic device (Johnston, 1992, Nature, 356, 152).

According to a further aspect, the invention relates to a pharmaceutical composition which comprises a protein or peptide, a nucleic acid molecule or a vector thereof according to the invention, together with pharmaceutically acceptable excipients, for the preventive or therapeutical treatment of tumors, in particular chronic myeloid leukemias and Ph-positive acute lymphoid leukemias. The pharmaceutical compositions can be administered through the parenteral, oral or topical route. The parenteral, intravenous or intramuscular routes are preferred. The procedure for the preparation of the pharmaceutical compositions are known to those skilled in the art; a detailed description can be found, for example, in Remington's Pharmaceutical Science, 17th and., Mack Publishing Company, Easton, Pa. (1985). The pharmaceutical compositions according to the invention are useful for the preventive or therapeutical treatment of tumors, in particular of chronic myeloid leukemia (CML) and Ph-positive acute lymphoid leukemia (ALL).

According to a preferred embodiment, the compositions are in the form of a vaccine, particularly suitable for the preventive vaccination of subjects with cancer susceptibility or for the immunotherapy of tumor patients. The amount of active ingredient in the pharmaceutical formulations according to the invention will be sufficient to trigger a humoral and/or cell-mediated immune response, preferably a CTL response against tumour cells. For peptides/proteins, said amount will depend on their physico-chemical properties, administration route, severity of the disease and on the conditions of the subject/patient. In principle, an amount ranging from 1 to 1000 μg will be sufficient, preferably 100 to 300 μg either in a single daily administration or in multiple administrations at different times. In case of DNA vaccination, the amount of DNA will be generally comprised between 100 and 1000 μg, preferably between 250 and 600 μg.

The general procedures for the preparation and use of vaccines are known to those skilled in the art (see for instance Paul, Fundamental Immunology, Raven Press, New York (1989) or Cryz, S. J., Immunotherapy and Vaccines, VCH Verlagsgesselshaft, 1991). Vaccines are preferably used in the form of injectable suspensions or solutions, or as solid or liposomal preparations. The immunologically active ingredients are mixed with one or more pharmaceutically acceptable excipients, such as emulsifiers, buffering agents or adjuvants which increase the vaccine effectiveness. The vaccine can be administered following a single or multiple dosage scheme. In case of multiple dosage, a variable number of separated doses are provided, each containing an antigen amount ranging from 1 to 1000 μg, followed by boosting doses at different times to keep or enhance the immune response. A prime-boosting approach, which comprises DNA priming followed by boosting with peptide and adjuvant or viral vectors (e.g. vaccinia vectors) or virosomes, is preferred.

In any case, the treatment regimen will depend on the patient's response and progression of the tumor disease.

Peptides/proteins and compositions thereof according to the invention can also be used in ex vivo methods. For example, antigen presenting cells or lymphocytes can be withdrawn from the patient and treated in vitro with the peptides, then re-introduced into the patient. Alternatively, patient cells can be transfected with vectors containing the sequences encoding for the tumors-specific antigens of the invention, propagated in vitro and re-introduced into the patient.

According to a preferred embodiment, the invention relates to an ex-vivo method for inducing a CTL or Th response against tumour cells bearing BCL/ABL fusion genes, which method comprises contacting the peptides of the invention with T lymphocytes or with antigen presenting cells (APC) under suitable conditions for their activation. Suitable APC cells comprise PBMC, dendritic cells, macrophages, or activated B cells. APCs can be genetically modified so as to express a particular HLA allele and cultured with T lymphocytes, optionally in the presence of one or more cytokines. Before being re-introduced into the patient, lymphocytes or APC cells can be purified, e.g. through an affinity column derivatized with a suitable ligand.

The invention further comprises APCs presenting a Bcl/Abl peptide on their surface. The peptide is preferably presented in the form of a complex with a specific HLA molecule. The invention also comprises an isolated lymphocyte, preferably a cytotoxic T lymphocyte, capable of recognizing and binding a complex consisting of an HLA molecule and a peptide of the invention. Specific cytotoxic T lymphocytic cell lines can be obtained by selection of cells that are activated by exposure to tumour cells harbouring Bcr/Abl fusion genes or proteins.

A further aspect of the invention relates to the use of APCs bearing an immunogenic peptide according to the invention, preferably a peptide selected from SEQ ID N. 1 and 5-17, or of autologous T cells capable of biding said peptide in a suitable HLA context, for the preparation of a therapeutical composition for the treatment of tumors, in particular of chronic myeloid leukemias and Ph-positive acute lymphoid leukemias.

Furthermore, the invention is directed to antibodies, fragments or derivatives thereof, which specifically recognize and bind the peptides or proteins of the invention. Methods for producing antibodies are known in the art (see e.g. Kohler and Milstein, Nature 256 (1975), 494, or J. G. R. Hurrel, Monoclonal Hybridoma Antibodies: Techniques and Applications, CRC Press Inc., Boco Raton, Fla., 1982). The antibodies according to the invention can be either monoclonal or polyclonal, or fragments of F(ab′)2, Fab, Fv or scFv type.

A further aspect of the invention relates to the use of immunogenic peptides, or nucleic acid sequences encoding them, for the preparation of a diagnostic composition. The latter can be used in molecular genetic, immunoblotting, immunocytochemistry or FACS techniques, for monitoring tumor progression, for example by quantitation of the peptide-specific T cells before, during and after tumor treatment.

### DETAILED DESCRIPTION OF THE INVENTION

The invention is based on the finding of novel hybrid transcripts generated by alternative splicing of the BCL and ABL genes involved in Philadelphia chromosome rearrangement. Because of a shift in the reading frame of the ABL gene located at 3′ end, the hybrid transcripts are translated into proteins that contain, at their carboxy termini, a number of “out of frame” amino acids which define unique sequences, and, at their amino termini, amino acid sequences encoded by BCR-gene exons.

The alternative splice variants were identified by i) carrying out a nested-RT-PCR with primer pairs complementary to different exons of BCR and ABL genes, ii) sequencing the 2^{nd}-step PCR amplification products and iii) comparing the obtained sequences with those available for BCR and ABL genes. By doing so, out of frame transcripts resulting from the alternative junction of exons 1, 13 and 14 of the BCR gene with exon 4 of the ABL gene were isolated. The transcripts—indicated as e1a4, b2a4 and b3a4—are found in nearly the totality of Ph-positive leukemias together with known major hybrid transcripts. In particular, samples containing the major hybrid transcripts b2a2, b3a2 and e1a2, were found to contain also b2a4, b3a4 and e1a4 transcripts, respectively. The alternative junctions of BCR exons with ABL exon 4 give rise to a shift in the reading frame of ABL gene (FIG. 5), which generates an early stop codon within ABL exon 5.

The production of hybrid fusion proteins (e.g. SEQ ID Nos: 2-4) carrying at the carboxy terminus an out of frame sequence of 112 amino acids (SEQ ID NO:1), along with the fusion proteins p190 and p210 generated by the major hybrid transcripts, was demonstrated using polyclonal antibodies against the whole out of frame portion (112 aa.). To that purpose, the complete cDNA (SEQ ID NO: 18) encoding the OOF portion was cloned in a plasmid vector and expressed in prokaryotic cells as a fusion protein which, after purification from bacterial proteins, was used for immunization.

The serum specific for the novel protein sequences was used in experiments of western blotting and immunoprecipitation of lysates from Ph-positive leukemic lines. The b3a4 and b2a4 alternative out of frame transcripts were found in the K-562 line, besides the major hybrid transcript b3a2, and, respectively, in CMLT-1 and JK lines, besides the major hybrid transcript b2a2. In addition, the TOM-1 line was found to contain the e1a4 alternative out of frame transcript, besides the major hybrid transcript e1a2. Lysates of leukemic cell lines negative for Bcr/Abl rearrangement, such as the HL60 line, were used as negative control.

The immunoblot of total extracts using a commercial anti-BCR monoclonal antibody, after immunoprecipitation with the anti-“out of frame” (anti-OOF) polyclonal serum, revealed the presence of a band at about 116 Kd in K-562 cells lysed with a detergent solution active on nuclear membranes. The same band is detected in western blots experiments using total K-562 extracts, while it is absent either in K-562 lysates obtained with a weak detergent solution able to extract only cytoplasmic proteins, or in HL-60 lysates whatever the detergent solution used (FIG. 7). These experimental results indicate that the alternative hybrid transcripts are translated into proteins and that the latter are mainly localized in the cell nucleus. The same results were confirmed by immunohistochemical assays on different Ph-positive leukemia lines (by RT-PCR) employing the anti-OOF serum, and by computer-assisted topology prediction, as described in the examples below.

Furthermore, the OOF portion of 112 aa. resulted immunogenic in outbred mice similar to humans as regards the MHC variability, and antigen-specific lymphocytes were identified after in vitro stimulation of PBMCs from a leukemic patient using peptides derived from the OOF sequence.

Altogether, the data demonstrate the effectiveness of the ‘out of frame’ proteins or peptides of the invention as tumor antigens to be used in the diagnostic, preventive and therapeutic treatment of tumors, in particular Ph-positive chronic myelogenous leukemia and acute lymphoblastic leukemia.

### DESCRIPTION OF THE FIGURES

FIG. 1—Localization of various breakpoints within the BCR and ABL genes, and schematic representation of the different fusion hybrid transcripts so far identified and of the corresponding predicted proteins.

FIG. 2—Schematic representation of the nested-PCR technique used in the identification of the alternative junctions b3a4, b2a4 and e1a4.

FIG. 3—1^{st} round of nested PCR: the major hybrid transcripts b2a2, b3a2 and e1a2 are detected.

FIG. 4—2nd round of nested-PCR: the alternative transcripts b2a4, b3a4 and e1a4 are detected together with the major hybrid transcripts b2a2, b3a2 and e1a2.

FIG. 5—Reading frame shift of the ABL gene as a result of alternative junctions between the BCR-gene exons 1, 13 and 14 and ABL-gene exon 4.

FIG. 6—Nucleotide and amino acid sequences of the out of frame (OOF) portion.

FIG. 7—Western blot of total lysates immunoprecipitated or not with anti-OOF, using anti-Bcr Mab for detection. Comparison between the p-210 positive CML K-562 cell line (positive control) and the promyelocytic leukemia HL60 cell line (negative control) using two different lysis buffers.

FIGS. 8A and B—Production of intracellular IL-2 in T cells (CD4+CD69+ or CD8+CD9+) activated by a mixture of OOF peptides specific for HLA-A3, determined by FACS analysis using a FITC-conjugated anti-IL2 antibody. The frequencies are expressed as percentage of the total T-cell population examined (CD4+ or CD8+).

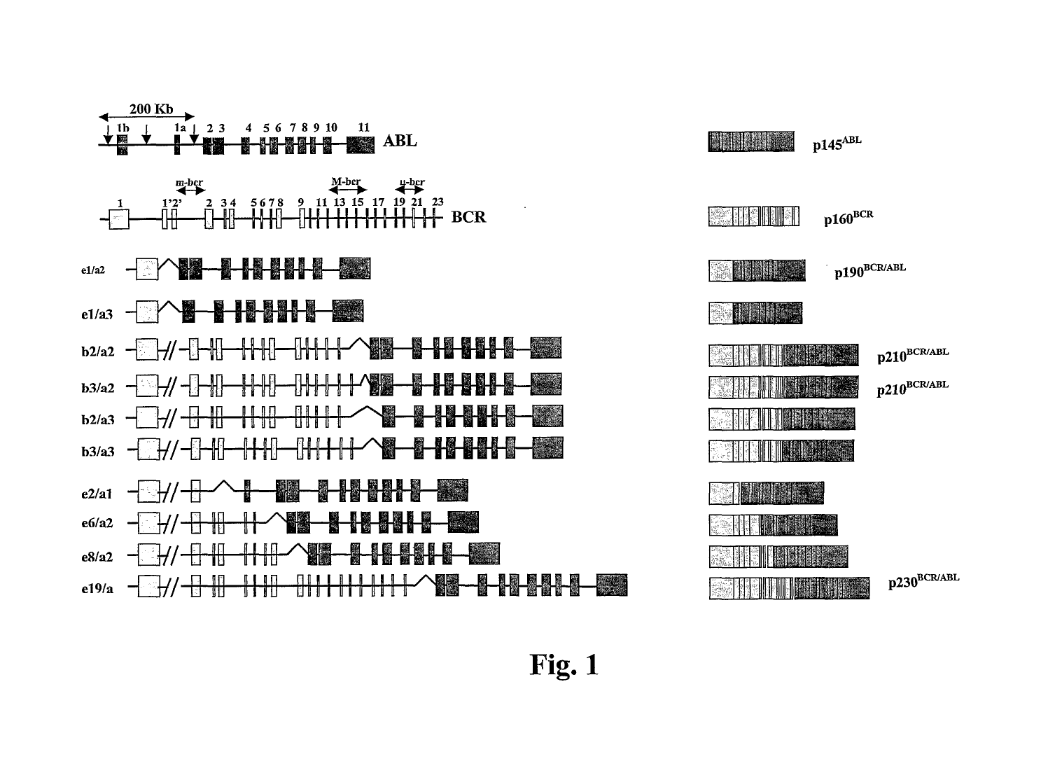

The following examples illustrate the invention in greater detail.

### EXAMPLE 1

### Identification of Alternative Hybrid Junctions Out of Frame between the BCR and ABL Genes Involved in the (t9;22)(q34;q11) Translocation, in Subjects Affected by Ph-Positive Chronic Myelogenous Leukemia or Acute Lymphatic Leukemia

Materials and Methods

A total of 50 medullar or peripheral blood samples from Ph-positive CML and ALL patients were analysed for the presence of hybrid transcripts out of frame (OOF) generated by alternative splicing between the BCR and ABL genes.

The p190-positive TOM-1 cell line (LLA) and 4 p210-positive LMC lines (K-562, KC122, CMLT1, JK-1) were used. Samples from normal subjects and the promyelocytic leukemic cell line HL60 negative for the t(9;22) rearrangement, were used as negative controls.

The total RNA was extracted according to modified Chomczynski P. and Sacchi N. (Anal. Biochem. 1987, 162:156) from the cell lines and from mononuclear cells isolated from BM or PB samples after separation on density gradient.

The subsequent reverse-transcription and two-step amplification reactions (nested PCR) were carried out according to the BIOMED 1 CONCERTED ACTION protocol of the European Commission for the standardization of the RT-PCR for the fusion gene transcripts (Van Dongen J J M et al., Leukemia 1999; 13: 1901-1928). In brief, 1 μg of total RNA was reverse-transcribed into c-DNA with the Perkin Elmer kit (Norwalk, C, USA), using the murine leukemia virus reverse transcriptase (MuLV Reverse Transcriptase). The use of random examers for reverse transcription allowed to use the same c-DNA in subsequent amplifications (nested PCR) with different primers complementary to the exons of the BCR and ABL genes.

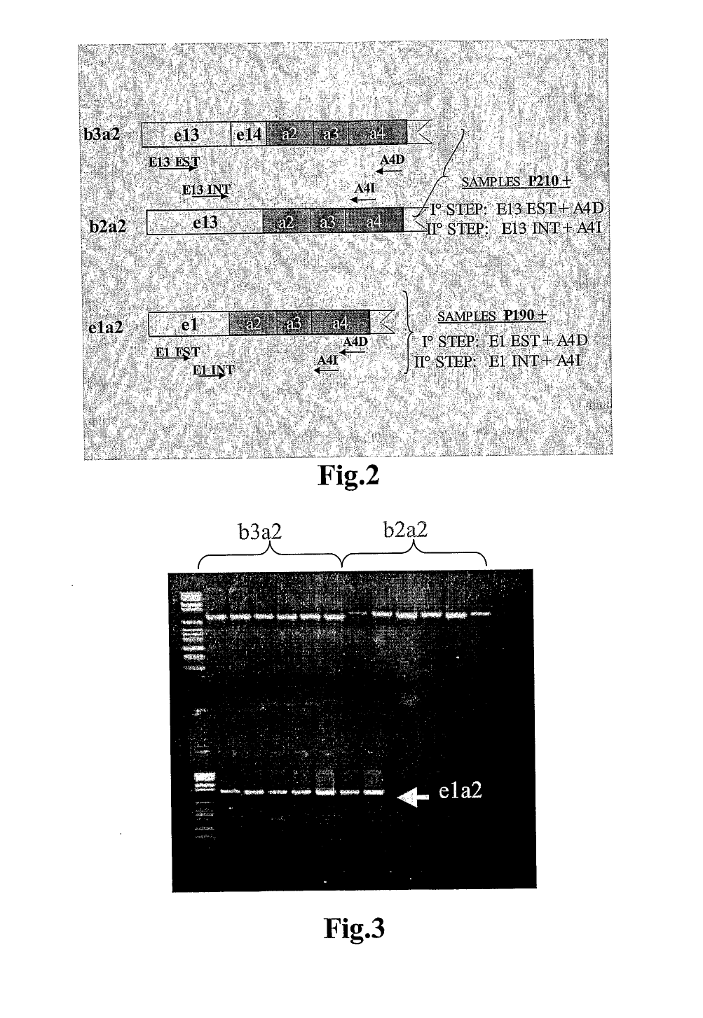

A schematic representation of the 2-step nested PCR is given in FIG. 2.

The PCR products were run on 2% agarose gel containing Ethidium bromide and the bands were visualized with a U.V. transilluminator.

Where the bands were absent, the c-DNA integrity was confirmed by amplification of the housekeeping ABL gene. The bands having molecular weights different from those of the major hybrid transcripts were extracted from the gel and purified on affinity column using the Nucleospin® Extract kit (Macherey-Nagel).

The purified products were directly sequenced from both ends, without cloning, using the ABI PRISM Big Dye Terminator v 3.1 (Perkin Elmer) Cycle Sequencing Kit and the oligonucleotides used in the second step of the nested PCR. Afterwards the products were purified using Ayto Seq G-50 columns (Amersham Pharmacia Biotec Inc., Piscataway, N.J.) and then separated by capillary electrophoresis with the ABI PRISM 3100 Genetic Analyzer (Applied Biosystems), and finally analysed with the ABI PRISM Gene Scan Analysis software.

The obtained sequences were compared to the corresponding sequences of BCR and ABL genes (available at Genebank acc. Code U07000/M24603 and U07563), in order to identify alternative hybrid transcripts and exclude any amplification artefact.

Results

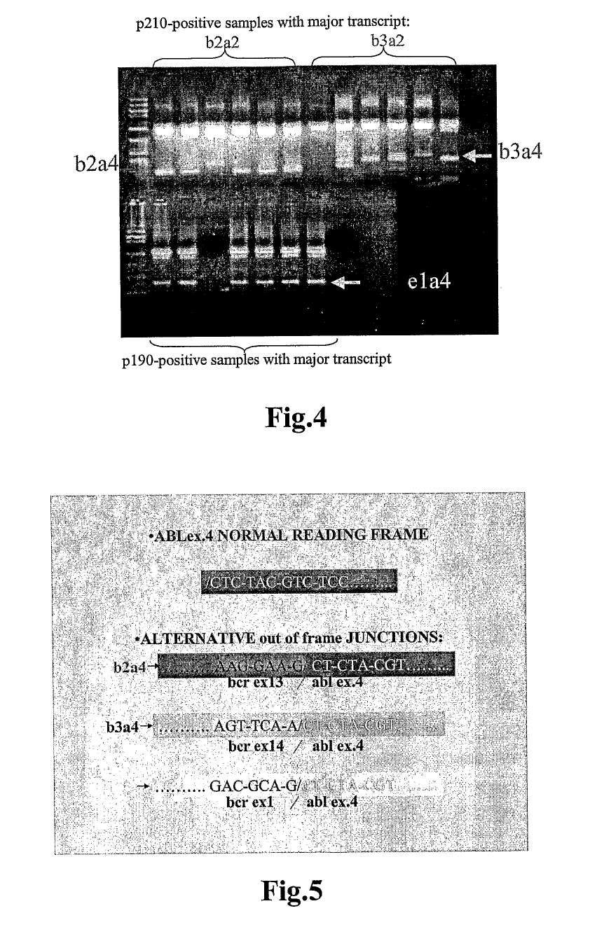

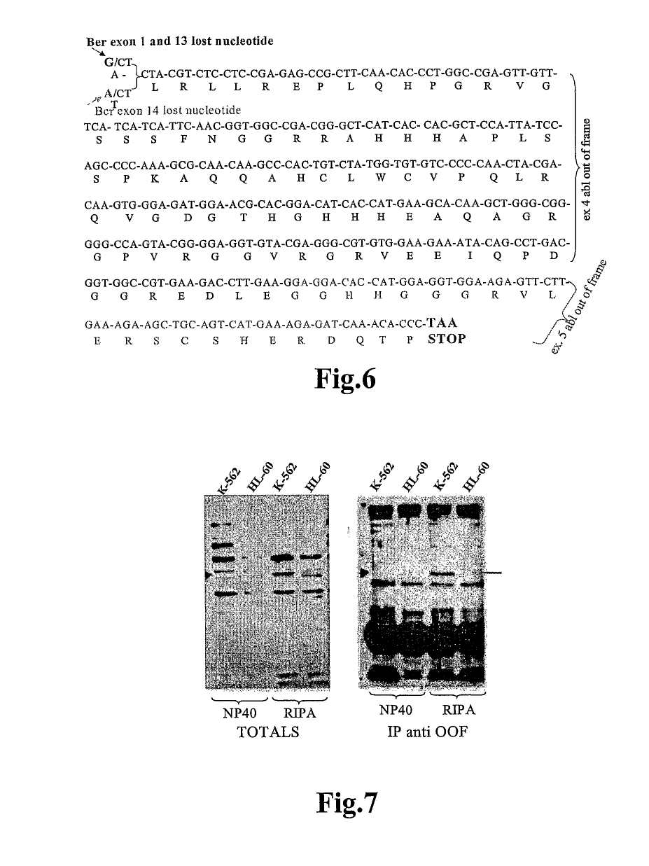

As shown in FIG. 3, after a first amplification step, all the examined samples presented only major BCR/ABL junctions (b3a2, b2a2, e1a2); at the second step, bands of different size and intensity appeared in many samples (FIG. 4). After sequencing, junctions involving exons 1, 13 and 14 of the BCR gene and exon 4 of the ABL gene were found. These junctions were present in more than 80% of the examined samples, together with the major hybrid transcripts, which are present in higher amounts. Specifically, the OOF transcripts b2a4, b3a4 and e1a4 were present with the major hybrid transcripts b2a2, b3a2 and e1a2.

The alternative junctions determine a shift in the reading frame of ABL exon 4 (“out of frame”), thus generating an early stop codon within the exon 5, suggesting that, besides the fusion proteins p210^{BCR/ABL} and p190^{BCR/ABL}, alternative BCR/OOF hybrid proteins may be present, carrying a 112 aa. portion at their carboxy terminus derived from a shift of the reading frame of the ABL gene.

### EXAMPLE 2

### Uniqueness and Tumor-Specificity of the Transcripts, and Immunogenicity of the Encoded OOF Proteins

No significant sequence-alignment resulted from the comparison of the protein portion encoded by Abl exons 4 and 5 in a shifted reading frame with protein databases using BLASTP (expect value: 10). Increasing the statistic significance threshold (expect value 20), only 7 alignments were obtained on restricted sequence portions, with scarce similarity and significance. For example, the alignment of 39 out of 113 amino acids with the nerve growth factor of Falco sparverius, resulted in a 30% sequence identity (12aa/39aa) and a 48% similarity (19aa/39aa); the alignment of 45 out of 113 amino acids with the “similar to growth arrest specific 2” protein of Mus musculus resulted in a 35% sequence identity (16aa/45aa) and 44% similarity (20aa/45aa).

The structural and functional analysis of the 116 Kda BCR/OOF protein was very interesting. The recombinant protein, analysed for putative domains or functional motives (Pattern and profile searches ExPASy server and others), revealed the presence of domains ascribable to Bcr (coiled coil region in the amino terminal region: dimerization domain; RhoGEF (Dbl-homologous domain); serine-threonine kinase activity domain; PH domain). The PSORT topology prediction analysis (PSORTII Server) indicated the presence, within the region encoded by Bcr exons, of the Bipartite Nuclear Targeting sequence KRANKGSKATERLKKKL, which can also be found using the ScanProsite at ExPASy (PROSITE reference: PS00015, PDOC00015).

Epitope Prediction and Different Human HLA

The sequence analysis in protein databases suggested that the OOF proteins contain amino acid sequences that are not present in normal cells and which therefore can be used as leukemia-specific genetic determinants for therapeutic anti-tumor approaches. It is known that, in order to induce an effective immune response, the tumor antigens should be processed and presented by (i.e. they should have affinity for) HLA molecules expressed on the cell surface.

The entire OOF sequence was analysed using methods for epitope prediction such as SYFPEITHI (database for MHC ligands and peptide motifs) and BIMAS (BioInformatics & Molecular Analysis Section), in order to identify T-cell specific epitopes to be used in immunotherapeutic approaches to antileukemic vaccination. The antigenic epitopes were selected for their affinity for the most representative HLA-class I alleles expressed in the group of leukemic subjects previously analysed by RT-PCR, namely HLA-A0201 and HLA-A3 (the same alleles are the most frequent in the caucasian population).

The following epitope candidates were identified:
HLA-A0201:

| | | |
|---|---|---|
| Pos. | AA. | Score BIMAS/SYFPEITHI |

| | | |
|---|---|---|
| 3 | LLREPLQHP | 0.018/19 |
| 37 | QQAHCLWCV | 121.64/16 |
| 72 | GVRGRVEEI | 0.078/21 |

HLA-A3:

| | | |
|---|---|---|
| Pos. | AA. | Score BIMAS/SYFPEITHI |

| | | |
|---|---|---|
| 2 | RLLREPLQH | 0.6/30 |
| 41 | CLWCVPQLR | 30/16 |
| 72 | GVRGRVEEI | 0.810/17 |
| 98 | RVLERSCSH | 0.045/25 |

The above peptides can be used for the preparation of specific monoclonal antibodies.

### EXAMPLE 3

### Cloning of the cDNA Encoding the Out of Frame Portion in a Prokaryotic Vector and Expression Thereof as Recocombinat Protein

The cDNA coding for the OOF portion was fused to the E. coli Maltose Binding Protein (MBP) gene and the fusion product was expressed in E. coli.

Enzyme restriction sites were introduced by PCR in the OOF cDNA ends, namely EcoRI and BamHI at the 5′ and 3′ ends, respectively. The fragment was subsequently cloned in correct frame in the corresponding EcoRI/BamHI sites of the pMAL-c2 plasmid vector (New England Biolabs, Inc, USA) at the MBP-gene 3′.

The insert-containing plasmid was sequenced using the ABI PRISM kit Big Dye Terminator Cycle Sequencing Ready Reaction (Perkin Elmer), as described in the example 1.

E. coli competent cells were transformed and the positive clones were examined for the presence of the genetic insert by extraction and digestion of the plasmidic DNA (Sambrook et al, Molecular Cloning: A laboratory Manual, Cold Spring Harbor Laboratory Press, Cold Spring Harbor, N.Y., 1989).

The MBP-OOF fusion protein was expressed in soluble form by induction of the positive bacterial cells with 0.3 mM isopropylthio-β-D-galactoside (IPTG) and subsequently purified by affinity chromatography on amilose resin according to the manufacturer's instructions.

To test the effectiveness and specificity of the antiserum, a GST-OOF fusion protein was prepared. To this end, the full-length cDNA encoding the OOF portion was amplified using synthetic oligonucleotides containing the restriction sites EcoRI and SalI and cloned in frame at the 3′ end of the Glutathione S-transferase (GST) gene in the EcoRI/SalI sites of the prokaryotic expression vector pGEX 5x-1 (Pharmacia Biotech). The fusion protein GST-OOF, the expression of which was induced with 0.1 mM IPTG, was finally purified on Glutathione Sepharose 4B (Pharmacia Biotech).

### EXAMPLE 4

### Protein Immunogenicity: Rabbit Immunization and Generation of Polyclonal Antibodies against the Entire OOF Portion

The anti-OOF rabbit polyclonal serum was obtained by rabbit immunization with the MBP-OOF fusion protein.

The rabbits were immunized with repeated i.m. injections of the purified fusion protein (300 γ) resuspended in Freund's complete adjuvant. Various blood samples were withdrawn at 15-20 day intervals and, the serum was separated and stored at −20° C. prior to be tested.

The antiserum specificity was demonstrated by immunoprecipitation and western blots of the GST-OOF fusion protein. The results were extremely positive, as the serum proved able to specifically immunoprecipitate the OOF portion from a total bacterial induction and to detect it by Western Blot even at high dilutions (1:20,000).

To purify the anti-OOF polyclonal antibodies from the rabbit serum, the GST-OOF fusion protein was dialized and then conjugated to the Sepharose 4B CnBr activated resin (Pharmacia Biotech). The protein conjugated to the resin was packed in the column and the latter was added with the pool of serum samples.

The antibodies adsorbed by the column were eluted with 0.1 M glycin-HCl pH 3, dialized and stored at 4° C.

### EXAMPLE 5

### Identification of a 116 Kd OOF Protein Using Anti-OOF Polyclonal Serum by Immunprecipitation of Ph-Positive K-562 Lysate

Materials and Methods

The K-562 cell line was selected among the Ph-positive leukemic cell lines previously assayed by RT-PCR for the presence of OOF transcripts. This cell line contains b3a2 as the major hybrid transcript and the out of frame b3a4 transcript. The HL60 leukemic cell line was used as negative control. The cells were cultured in complete RPMI 1640 added with 10% FBS.

After two washings with cold PBS, the cells were resuspended in lysis solution containing the protease inhibitors (aprotinin, leupeptin and pepstatin).

Two different lysis buffers were used, one for the extraction of cytoplasmic proteins, containing 150 mM NaCl, 50 mM Tris HCl pH 8, 1 mM EDTA, 1% Nonidet P-40, and a stronger one (RIPA buffer) able to lyse the nuclear membrane containing 50 mM Tris pH 7.5, 150 mM NaCl, 1% Triton X-100, 1% sodium deoxycholate and 0.1% SDS. After 30′ on ice the extracts were centrifuged and their protein content was determined with Bio-Rad Assay (BIORAD). 6 mg of each protein extract were immunoprecipitated with the polyclonal anti-OOF serum, as above described. Then Sepharose Prot A was boiled in SDS 1% to elute the bound proteins; the eluate and 100 μg total protein extract were separated by SDS electrophoresis on 6% polyacrylamide gel and blotted to nitrocellulose membrane (Hybond C, Amersham Pharmacia). The membranes were saturated with TBS—5% BSA and incubated overnight at 4° C. in TBS 1% BSA containing primary anti BCR antibody (Santa Cruz) diluted 1:500 in TBS % BSA. This antibody is generated against an epitope localized at the NH2-terminus of BCR, and recognizes the BCR protein and any hybrid BCR/ABL fusion proteins. After washing, the filters were incubated with peroxidase conjugated anti mouse antibody (Amersham Pharmacia) for 2 hr at room temperature; detection was performed with chemiluminescent substrate Lite Ablot (Celbio).

Results

As shown in FIG. 7, the immunoblotting with anti-BCR antibody of the total extracts immunoprecipitated with the anti OOF polyclonal serum, reveals a protein band having a molecular weight around 116 Kda in the RIPA buffer lysate of the K-562 line. The same band is present in the total extract, whereas it cannot be detected either in K-562 cells lysed with NP-40, or in HL-60 cells whatever the lysis buffer used.

This result demonstrates that the OOF transcripts are translated into proteins and that the latter are localized in the nucleus, as predicted by the analysis of the protein-structure.

### EXAMPLE 6

### Immunohistochemical Localization of the OOF Protein Sequence in the Nucleus of Ph-Positive Cells

The Ph1-negative HL60 cell line and the Ph1-positive K562, JK, TOM-1, CMLT-1 leukemic cell lines were immunohistochemically stained. 10⁵ cells were centrifuged by citospin (1000 g/10 min) on a glass slide and fixed in 98% ethanol. Each experiment was carried out on a minimum of 6 samples and repeated at least four times.

The endogen peroxidase activity was inhibited by treating the slides with 3% hydrogen peroxide for 10 minutes at room temperature.

Each cell line was treated with anti-OOF rabbit immune serum, pre-immune serum, anti-Bcr (control for cytoplasmic positivity) and anti-Ki67 (control for nuclear positivity) antibodies. After washings with TBS buffer, slides were incubated for 2 hours at room temperature with pre-immune and immune rabbit serum diluted 1:50. In addition, the slides were incubated with anti-Bcr rabbit polyclonal antibody at 1:200 dilution (Santa Cruz Biotechnology) and with the anti-Ki67 mouse monoclonal, clone MIB-1 (Dako) diluted 1:50. Slides were then incubated with ABC Staining System (Santa Cruz Biotechnology) biotin-conjugated secondary antibodies according to the manufacturer's instructions. Bound antibodies were visualized by incubating with diaminobenzidine (DAB) solution and counter-stained with hematoxylin.

Immunoreactivity Analysis

The slides treated with the immune serum showed an intense and homogeneous nuclear staining for cell lines K562, JK, TOM-1 and CMLT-1. No staining was detected in the slides treated with pre-immune serum. Likewise, no immunostaining was detected in HL60 cells treated with either pre-immune or immune serum.

### EXAMPLE 7

### Construction of a Minigene for Eukaryotic Expression Using the Natural Sequence or a Codon-Usage Modified Sequence

Plasmid vectors were assembled on the backbone of the pRC110 plasmid expressing murine IL2 in a transcriptional cassette, and a second cassette containing unique sites for directional cloning of the antigen.

| | |
|---|---|
| Clone name: | pRCBcr/Abl 112AA |
| Host bacterial strain: | Escherichia coli DH5α |
| Eukaryotic bacterial vector: | pRC110 |
| Antibiotic resistance: | 50 μg/ml ampicillin |
| Description: | cloning of the in frame 5′-3′NheI-NotI |
| | fragment (112 aa; 345 bp) |

Below, the putative out of frame mRNA obtained by alternative splicing of the genes involved in the Bcr/Abl rearrangement, specifically Bcr exons 1, 13 or 14 and Abl exon 4. In bold character the exon-5 fragment extending up to the stop codon generated by the shift of the reading frame of ABL.

The 5′ portion sequentially comprises the NheI cloning site, the Kozak consensus sequence for translation initiation, the ATG translation initiation codon and the 112 OOF coding sequence.

5′-GCTAGC GCC ATG CTA CGT CTC CTC CGA GAG CCG CTT CAA CAC
NheI Kozak MET leu arg leu leu arg glu pro leu gln his
CCT GGC CGA GTT GGT TCA TCA TCA TTC AAC GGT GGC CGA CGG
pro gly arg val gly ser ser ser phe asn gly gly arg arg
GCT CAT CAC CAC GCT CCA TTA TCC AGC CCC AAA GCG CAA CAA
ala his his his ala pro leu ser ser pro lys ala gln gln
GCC CAC TGT CTA TGG TGT GTC CCC CAA CTA CGA CAA GTG GGA
ala his cys leu trp cys val pro gln leu arg gln val gly
GAT GGA ACG CAC GGA CAT CAC CAT GAA GCA CAA GCT GGG CGG
asp gly thr his gly his his his glu ala gln ala gly arg
GGG CCA GTA CGG GGA GGT GTA CGA GGG CGT GTG GAA GAA ATA
gly pro val arg gly gly val arg gly arg val glu glu ile
CAG CCT GAC GGT GGC CGT GAA GAC CTT GAA GGA GGA CAC CAT
gln pro asp gly gly arg glu asp leu glu gly gly his his
GGA GGT GGA AGA GTT CTT GAA AGA AGC TGC AGT CAT GAA AGA
gly gly gly arg val leu glu arg ser cys ser his glu arg
GAT CAA ACA CCC TAA GCGGCCG-3′
asp gln thr pro STOP NotI

### EXAMPLE 8

### DNA Vaccination for Immunization Purpose and for the Production of Polyclonal Antibodies Against Specific Peptides

The studies of immunogenicity and of immune response were conducted in outbred murine models, similar to humans as regards their MHC variability. The constructs were used to immunize outbred Swiss/CD1 mice so as to verify the immunogenicity of the protein.

Protocol used for DNA vaccination with different constructs:

MICE: CD1 (ex SWISS)

GENDER: female

AGE: 6-8 weeks

pRC 100: control plasmid vector

pRC Bcr/Abl 112 AA: long out of frame plasmid vector

Injection site: rectus femoris muscle

DNA: 100 μg/50 μl

| | | | |
|---|---|---|---|
| | | t = 7 | |
| t = 0 weeks | t = 5 weeks | weeks | t = 8 weeks |

| | serum | DNA inj | serum | DNA inj | serum | Serum |
|---|---|---|---|---|---|---|
| AE | 130 μl | pRC100 | 210 μl | pRC100 | 120μ | 400μ |
| AF | 12 | pRC100 | 25 | pRC100 | 25 | 50 |
| AG | 15 | pRC100 | 135 | pRC100 | 100 | 85 |
| AH | 40 | pRC100 | 30 | pRC100 | 60 | |
| AI | 150 | pRC100 | 40 | pRC100 | 75 | 27 |
| AL | 110 | pRC112aa | 75 | pRC112aa | 35 | 60 |
| AM | 60 | pRC112aa | 45 | pRC112aa | 150 | 70 |
| AN | 25 | pRC112aa | 70 | pRC112aa | 160 | 4 |
| AO | 35 | pRC112aa | 105 | pRC112aa | 40 | |
| AP | 75 | pRC112aa | 23 | pRC112aa | 160 | 55 |
| AQ | 130 | pRC112aa | 50 | pRC112aa | 50 | 105 |
| AR | 105 | pRC112aa | 47 | pRC112aa | 50 | 55 |
| AS | 90 | pRC112aa | 140 | pRC112aa | 10 | 110 |
| AT | 20 | pRC112aa | 44 | pRC112aa | 180 | 28 |
| AU | 15 | pRC112aa | 25 | pRC112aa | 25 | 30 |

Test ELISA

| | DILUTIONS | | | | | |
|---|---|---|---|---|---|---|
| | 1:10 | | | 1:20 | | |
| | | MBP- | | | MBP- | |
| SERUM* | MBP | OOF | Δ | MBP | OOF | Δ |
| AQ (pre-immune) | 0.161 | 0.121 | — | 0.122 | 0.111 | — |
| AE (empty plasmid) | 0.299 | 0.121 | — | 0.301 | 0.108 | — |
| AG (empty pl.) | 0.309 | 0.188 | — | 0.315 | 0.221 | — |
| AI (empty pl.) | 0.194 | 0.170 | — | 0.166 | 0.153 | — |
| AM (pl. + OOF) | 0.823 | 0.294 | — | 0.689 | 0.256 | — |
| AN (pl. + OOF) | 0.256 | 0.149 | — | 0.265 | 0.182 | — |
| AO (pl. + OOF) | 0.085 | 0.113 | +0.028 | 0.086 | 0.114 | +0.028 |
| AP (pl. + OOF) | 0.250 | 0.411 | +0.161 | 0.192 | 0.318 | +0.126 |
| AS (pl. + OOF) | 0.404 | 0.142 | — | 0.432 | 0.134 | — |
| AT (pl. + OOF) | 1.010 | 0.833 | — | 0.935 | 0.838 | — |

| CONTROLS | MBP | MBP-ABL | 38C13 IgM Id |
|---|---|---|---|
| Secondary Ab only | 0.055 | 0.052 | N.D. |
| anti 38C13 serum IgM Id | N.D. | N.D. | 0.636 |
| (1:300) | | | |
| Secondary Ab only | N.D. | N.D. | 0.090 |

### EXAMPLE 9

### Immunogenicity of the OOF Protein: Identification of Antigen-Specific Lymphocytes after PBMC Stimulation in vitro with OOF Peptides

Antigen-specific T cells can be identified after in vitro restimulation with the specific antigen. Upon antigenic recognition, T cells undergo proliferation and cytokine secretion.

The presence of such cells indicates the immunogenicity of the protein or peptide that induced proliferation, allowing the use thereof in vaccination protocols.

The possibility of monitoring and quantitating the specific T-cell response by means of rapid tests before, during and after treatment is important for the development of anti-tumor vaccination approaches.

In this experiment, T cells specific for the OOF peptides were identified by cytofluorimetric detection of intracellular cytokines. The latter are produced by ex vivo stimulation of PMBCs from a leukemic patient, using an appropriate peptide mixture having predicted HLA-A3 binding according to BIMAS and SYFPEITHI software.

Materials and Methods

2×10⁶ PBMCs from a HLA-A3 patient affected by LMC were incubated at a concentration of 1×10⁶ cells/ml with or without a solution containing 4 different HLA-A3 binding peptides at 10 μg/ml (CLWCVPQLR, RLLREPLQH, RVLERSCSH, GVRGRVEEI). The superantigen staphylococcal enterotoxin B (SEB) was used as positive control. The toxin, in the presence of a co-stimulatory signal (anti CD28), stimulates the production of cytokines from CD4and CD8. After 1 hr, each sample was added with 20 μg brefeldine A and after additional 16 hrs the cells were washed and the surface antigens labeled with APC-, PE- and PerCP-conjugated monoclonal antibodies against CD8, CD69 and CD4, respectively. Subsequently the cells were fixed and permeabilized prior to addition of the FITC-conjugated monoclonal antibodies against IL-2, TNFα and the PE-conjugated monoclonal antibodies against IFNγ, IL-4.

FACScalibur cytofluorimeter and the Cell Quest software were used for data acquisition (25,000 events per each analysis).

Results

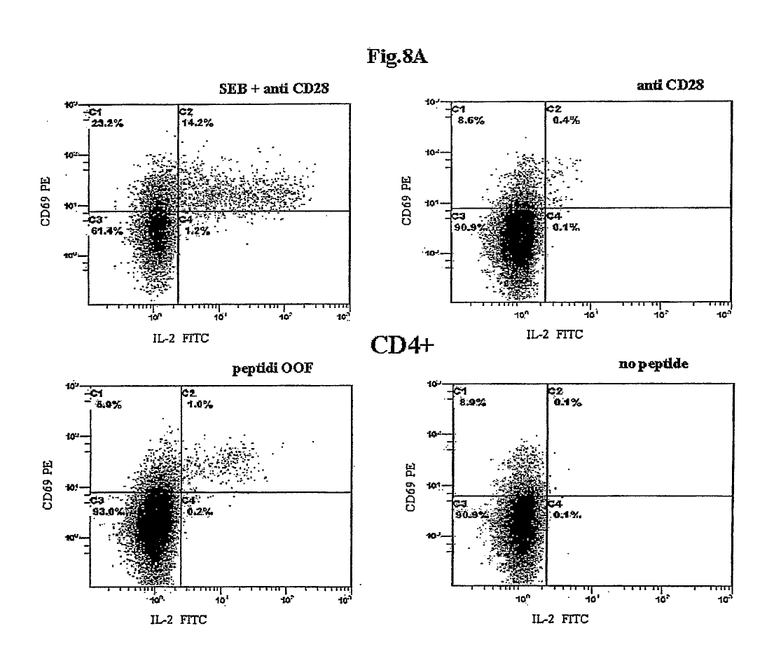

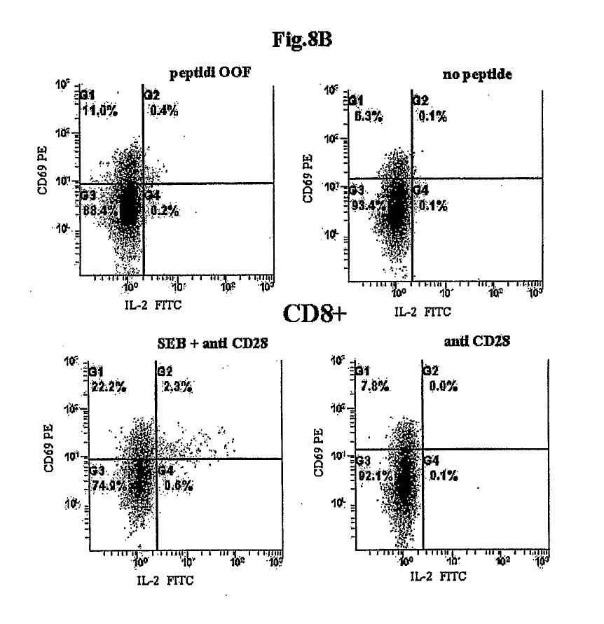

As shown in FIG. 8A, the mixture of 4 OOF peptides induces the accumulation of intracellular IL-2 in activated CD4+ and CD8+ T cells. These cells are identified by cytofluorimetric detection of the cell markers CD69 (indicative of cell activation) and with anti-IL-2. In particular, the frequency of CD4 cells producing IL-2 is 1% of the total CD4+ T population. In non stimulated PBMCs (i.e. in the absence of peptides) only 0.1% of the CD4+ cells produce IL-2, thus the observed increase (0.9%) following to peptide incubation appears specific. The fact that 14.2% of CD4+ cells produces IL-2 after stimulation with SEB+ anti CD28 (positive control) confirms the validity of this analysis. The CD8+ cells producing IL-2 (FIG. 8B) were only 0.4% of total CD8+ T cells. Hence the amount of specific CD8+ T cells producing IL-2 is 0.3% compared with CD8+ T cells unstimulated by the peptides.

### EXAMPLE 10

### Epitope Prediction and Candidate Selection for Abl Out Of Frame Sequence Optimization

Sequence:

LRLLREPLQHPGRVGSSSFNGGRRAHHHAPLSSPKAQQAHCLWCVPQLRQ
VGDGTHGHHHEAQAGRGPVRGGVRGRVEEIQPDGGREDLEGGHHGGGRVL
ERSCSHERDQTP

In order to identify one or more candidate epitopes for the MHC I optimization of the above indicated sequence, the HLA alleles with the highest frequency in Caucasian population were analysed with the SYFPEITHI software, in particular:

HLA-0210 (50% frequency)

HLA-03

HLA-B2705 (15%)

HLA-B0702 (11%)

HLA-B5101

the following epitope candidates were identified:
HLA-0201:

| | | |
|---|---|---|
| Pos. | AA. | Score |

| | | |
|---|---|---|
| 30 | PLSSPKAQQ | 10 |
| 34 | PKAQQAHCL | 10 |
| 64 | AGRGPVRGG | 10 |
| 68 | PVRGGVRGR | 10 |
| 98 | RVLERSCSH | 10 |
| 99 | VLERSCSHE | 10 |

HLA-03:

| | | |
|---|---|---|
| Pos. | AA. | Score |

| | | |
|---|---|---|
| 30 | PLSSPKAQQ | 16 |
| 31 | LSSPKAQQA | 10 |
| 64 | AGRGPVRGG | 8 |
| 68 | PVRGGVRGR | 5 |
| 98 | RVLERSCSH | 25 |

HLA-B2705:

| | | |
|---|---|---|
| Pos. | AA. | Score |

| | | |
|---|---|---|
| 30 | PLSSPKAQQ | 4 |
| 34 | PKAQQAHCL | 13 |
| 64 | AGRGPVRGG | 7 |
| 68 | PVRGGVRGR | 6 |
| 98 | RVLERSCSH | 17 |
| 99 | VLERSCSHE | 17 |

HLA-B0702:

| | | |
|---|---|---|
| Pos. | AA. | Score |

| | | |
|---|---|---|
| 30 | PLSSPKAQQ | 6 |
| 34 | PKAQQAHCL | 11 |
| 64 | AGRGPVRGG | 10 |
| 68 | PVRGGVRGR | 5 |
| 98 | RVLERSCSH | 1 |
| 99 | VLERSCSHE | 0 |

HLA-B5101:

| | | |
|---|---|---|
| Pos. | AA. | Score |

| | | |
|---|---|---|
| 30 | PLSSPKAQQ | 0 |
| 34 | PKAQQAHCL | 7 |
| 64 | AGRGPVRGG | 10 |
| 68 | PVRGGVRGR | 4 |
| 98 | RVLERSCSH | 4 |
| 99 | VLERSCSHE | 3 |

In the epitope prediction analysis for major HLA alleles present in the Caucasian population, the epitope in position 64-72 of the 112 aa. OOF, among the various HLA alleles examined, scores closely to the optimization value (=10). This peptide is therefore the best candidate for MHC I optimization.

### EXAMPLE 11

### Cytokine-Response of CML Patients upon PBMC Stimulation with OOF Derived HLA-A2 or HLA-A3 Binding Peptides

Intracellular cytokine-production by CD8+ T cells were measured in HLA-A2 or HLA-A3 CML patients and healthy subjects. We decided, for PBMC peptide stimulation, to pull together three peptides with major score for HLA-A3 (mix A3), two with major score for HLA-A2 (mix A2), and to use alone one with a good score for both type of HLA(A2/A3). We detected, into CML patients, between 0.2% and 1.3% of OOF-peptide-specific IFNγ+CD8+ T cells, after subtracting IFNγ+ CD8+ T cells frequency of unstimulated PBMC (see FIG. 7 for patients data and FIG. 8 for plots from one representative sample), whereas no positive response was seen in PBMC coming from healthy donors. Patients and healthly donor IL-2 and IFNγ CD8+ T cells production following stimulation with SEB and anti-CD28 confirmed method validity.

Methods

Cytokine Flow Cytometry (CFC) Assay

For CFC assay we collected by ficoll PBMC coming from HLA-A2 and HLA-A3 healthy donors and Ph-positive CML patient in cytogenetic complete remission. Patients were selected on the basis of bcr/abl alternative splicing presence and type of HLA class I molecules. 2×10⁶ cells, resuspended in serum-free medium (x-vivo, Biowittaker) were incubated at 37° C. in a humidified 5% C02 atmosphere for 14-16 h with a 10 μg/ml solution consisting of HLA-A2 or A3 specific binding predicted OOF peptides. After at least 1 h from the beginning of the culture, 10 μg/ml brefeldin A (Sigma) was added. In the same culture conditions were also included, as negative control, unstimulated PBMC whereas PBMC stimulated with a superantigen, SEB (staphylococcal enterotoxin B; 5 μg/ml; Sigma, St. Louis, Mo.) together with anti CD28 purified monoclonal antibody (1 μg/ml) as costimulatory signal was used as positive control (15). The following day, cells were washed in PBS and stained for 30′ at 4° C. in the dark with anti human CD8 APC (CALTAG Laboratories) then fixed and permeabilized using FIX & PERM kit (CALTAG) according manufacturer's instruction. Finally cells were stained with MoAb anti IL-2 FITC and anti IFN-γ PE (all from Caltag Laboratories) for 30′ at 4° C. in the dark. For all samples were acquired at least 60000 total events on a Becton Dickinson FACS Calibur flow cytometer using the Cell Quest software for the following analysis.

1. An isolated tumor-specific immunogenic peptide having sequence SEQ ID NO: 1, or an immunogenic fragment thereof.

2. A tumor-associated protein containing the peptide of claim 1.

3. A tumor associated protein according to claim 2, which consists of two portions, respectively containing the carboxy and amino ends, the former portion being encoded by one or more exons of the BCR gene (GenBank U07000), the latter consisting of SEQ ID NO: 1.

4. A protein according to claim 3, which is selected from the group consisting of SEQ ID NO: 2, 3, 4.

5. An immunogenic peptide fragment according to claim 1, which is selected from the group consisting of SEQ ID NO: 5, 6, 7, 8, 9, 10, 11, 12, 13, 14, 15, 16, 17.

6. A nucleic acid molecule encoding a peptide or a protein according to claim 1.

7. A nucleic acid molecule according to claim 6, coding for SEQ ID NO: 1, having sequence SEQ ID NO. 18.

8. An expression or cloning vector containing a nucleic acid molecule according to claim 6.

9. An eukaryotic or prokaryotic cell carrying the vector of claim 8.

10. A monoclonal or polyclonal antibody able to recognize and specifically bind a peptide or protein according to claim 1.

11. A pharmaceutical or diagnostic composition containing a peptide or a protein according to claim 1.

12. A composition according to claim 11, which is in the form of a vaccine.

13. A method in vitro for inducing an immune response against tumors harbouring BCR/ABL fusion genes, which comprises contacting a peptide or a protein according to claim 1 with T lymphocytes or with APCs in suitable conditions for their activation.

14. A complex formed by a peptide according to claim 5 and by a HLA molecule.

15. An isolated T lymphocyte which is able to bind a peptide or a complex according to claim 14.

16. An isolated APC presenting a peptide of claim 1 on its surface.

17-18. (canceled)

19. A pharmaceutical or diagnostic composition containing a nucleic acid according to claim 6.

20. A pharmaceutical or diagnostic composition containing a vector according to claim 8.

21. A method of treating tumours comprising administering to a subject in need thereof an effective amount of T-lymphocyte according to claim 15.

22. The method according to claim 21, wherein the tumours are Ph-positive chronic myeloid leukaemia, and acute lymphoblastic leukaemia.

23. A method of treating tumours comprising administering to a subject in need there of an effective amount of an APC according to claim 16.

24. The method according to claim 23, wherein the tumours are Ph-positive chronic myeloid leukaemia, and acute lymphoblastic leukaemia.


SEQUENCE LISTING (18 sequences)

SEQ ID NO: 1 - protein, 112 aa, Homo sapiens
LRLLREPLQHPGRVGSSSFNGGRRAHHHAPLSSPKAQQAHCLWCVPQLRQVGDGTHGHHHEAQAGRGPVRGGVRGRVEEIQPDGGREDLEGGHHGGGRVLERSCSHERDQTP

SEQ ID NO: 2 - protein, 539 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDAALRLLREPLQHPGRVGSSSFNGGRRAHHHAPLSSPKAQQAHCLWCVPQLRQVGDGTHGHHHEAQAGRGPVRGGVRGRVEEIQPDGGREDLEGGHHGGGRVLERSCSHERDQTP

SEQ ID NO: 3 - protein, 1015 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTELLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEALRLLREPLQHPGRVGSSSFNGGRRAHHHAPLSSPKAQQAHCLWCVPQLRQVGDGTHGHHHEAQAGRGPVRGGVRGRVEEIQPDGGREDLEGGHHGGGRVLERSCSHERDQTP

SEQ ID NO: 4 - protein, 1040 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTELLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSTLRLLREPLQHPGRVGSSSFNGGRRAHHHAPLSSPKAQQAHCLWCVPQLRQVGDGTHGHHHEAQAGRGPVRGGVRGRVEEIQPDGGREDLEGGHHGGGRVLERSCSHERDQTP

SEQ ID NO: 5 - protein, 9 aa, Homo sapiens
PLSSPKAQQ

SEQ ID NO: 6 - protein, 9 aa, Homo sapiens
PKAQQAHCL

SEQ ID NO: 7 - protein, 9 aa, Homo sapiens
AGRGPVRGG

SEQ ID NO: 8 - protein, 9 aa, Homo sapiens
PVRGGVRGR

SEQ ID NO: 9 - protein, 9 aa, Homo sapiens
RVLERSCSH

SEQ ID NO: 10 - protein, 9 aa, Homo sapiens
VLERSCSHE

SEQ ID NO: 11 - protein, 9 aa, Homo sapiens
LSSPKAQQA

SEQ ID NO: 12 - protein, 9 aa, Homo sapiens
LLREPLQHP

SEQ ID NO: 13 - protein, 9 aa, Homo sapiens
QQAHCLWCV

SEQ ID NO: 14 - protein, 9 aa, Homo sapiens
GVRGRVEEI

SEQ ID NO: 15 - protein, 9 aa, Homo sapiens
RLLREPLQH

SEQ ID NO: 16 - protein, 9 aa, Homo sapiens
CLWCVPQLR

SEQ ID NO: 17 - protein, 9 aa, Homo sapiens
GVRGRVEEI

SEQ ID NO: 18 - DNA, 339 bp, Homo sapiens
ctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaa